(19) United States
(12) Patent Application Publication
(10) Pub. No.: US 2006/0034813 A1
(43) Pub. Date: Feb. 16, 2006

(54) Expression of proteins in cord blood-derived endothelial cells

(76) Inventors: Christian Herder, Dusseldorf (DE); Torsten Tonn, Frankfurt am Main (DE); Manuel Grez, Heidelberg (DE); Erhard Seifried, Konigstein (DE)

Correspondence Address: CERMAK & KENEALY LLP, 515 E. BRADDOCK RD, ALEXANDRIA, VA, 22314, US

(21) Appl. No.: 11/244,087

(22) Filed: Oct. 6, 2005

Related U.S. Application Data
(63) Continuation of application No. PCT/EP04/03998, filed on Apr. 15, 2004.

(30) Foreign Application Priority Data
Apr. 15, 2003 (EP) 03008076.6

(51) Int. Cl. A61K 48/00; C12N 5/08; C12N 15/867

(52) U.S. Cl. 424/93.21; 435/456; 435/372

(57) ABSTRACT

The invention provides a population of mature endothelial cells derived from human cord blood. The cells can be transduced with DNA encoding a therapeutically effective protein such as a blood coagulation factor. The cells are useful in a method for the production of a blood coagulation factor. They can further be used in hemophilia A or B gene therapy.

This application is a Continuation of, and claims priority under 35 U.S.C. §120 to, international application PCT/EP2004/003998, filed Apr. 15, 2004, and claims priority under 35 U.S.C. 119 to European application number 03008076.6, filed Apr. 15, 2003, the entirety of both of which is incorporated by reference.

### BACKGROUND OF THE INVENTION

1. Field of the Invention

The invention relates to a method for the production of a protein, e.g., a blood coagulation factor. The method comprises culturing endothelial cells derived from human cord blood and isolating the desired protein from the cell culture medium. The invention further relates to a population of human endothelial cells transduced with a vector encoding a protein, e.g., a blood coagulation factor. The cells can be used in the method for the production of a protein or in a gene therapy protocol.

2. Brief Description of the Related Art

Hemophilia A is caused by insufficient levels or even the complete absence of functional coagulation factor VIII (FVIII) in the circulation. It is an X chromosome-linked bleeding disorder that affects 1 in 5,000 to 10,000 males. FVIII is an essential component of the intrinsic pathway of the blood coagulation cascade, where it serves as a cofactor for activated factor IX within a membrane-bound complex (Xase complex) that activates factor X, which in its turn participates in conversion of the zymogen prothrombin into the enzyme thrombin. FVIII is synthesized as a 2332 amino acid residue molecule (˜300 kDa) consisting of three homologous A domains, two homologous C domains, and a unique B domain arranged in the order A1-A2-B-A3-C1-C2. FVIII is processed by multiple intracellular cleavages within the B domain, and at the B-A3 junction, to a heterodimer consisting of Me²⁺-linked light and heavy chains. The heavy chain is comprised of the A1 (1-336), A2 (373-740) and B domains (741-1648) and the light chain includes the A3 (1649-2019), C1 (2020-2172) and C2 (2173-2332) domains. In the circulation, FVIII is tightly bound to von Willebrand factor (VWF), a protein required for maintaining the normal FVIII level in plasma. VWF prevents premature formation of the Xase complex and protects FVIII from inactivation by activated protein C, activated factor IX (FIXa) and activated factor X (FXa). VWF deficiency in both humans and animals has been shown to lead to a secondary deficiency of FVIII, thus suggesting that VWF also prevents FVIII from accelerated clearance.

The main symptoms of hemophilia A are bleedings into joints, muscles and internal organs that can occur spontaneously and be life-threatening (Mannucci and Tuddenham, 2001; see infra for bibliographic data of the references). Based on the residual activity of FVIII in plasma, hemophilia A is categorized as severe (<1% of normal activity), moderate (1-5%) and mild (5-30%).

Hemophilia B occurs in about 1 of 25,000 males. It is characterized by the deficiency of the serine protease factor IX (Christmas factor). This 415 amino acid polypeptide is synthesized in the liver as a 56 kDa glycoprotein. In order to attain its proper function a posttranslational carboxylation step is required which only occurs in the presence of vitamin K.

Hemophilia A and B patients are currently treated by intravenous infusions of plasma-derived or recombinant FVIII and FIX, respectively (Mannucci and Giangrande, 2000). Although this substitution therapy has become relatively safe and efficacious during the last decade, there are several drawbacks such as the inconvenience of lifelong infusions and the potential transmission of infectious diseases by FVIII concentrates (Hoots, 2001; Teitel, 2000; White II et al., 2000; VandenDriessche et al., 2001). Due to a relatively short half-life of FVIII in the circulation (12-14 h), prophylactic treatment of hemophilia A requires repeated—up to three times a week—infusions of FVIII preparations.

The use of non-human cell lines for the production of recombinant factor VIII encountered certain disadvantages. The major limitation in production of recombinant FVIII is low yield from FVIII expressing cells, which is two orders of magnitude lower than that for other proteins.

Several in vivo studies for the gene therapy of hemophilia A with viral and non-viral vectors were performed (reviewed in White II, 2001; Chuah et al., 2001; Greengard and Jolly 1999, VandenDriessche et al., 2001; Kaufman, 1999). However, there remain concerns over the safety of these approaches. Potential side effects include adverse immunological reactions, vector mediated cytotoxicity (Yang et al., 1996; Lozier et al., 1999) and germ-line transmission.

Under physiological conditions FVIII synthesis mainly occurs in hepatocytes and liver sinusoidal endothelial cells (Wion et al., 1985; Zelechowska et al., 1985; Do et al., 1999; Hollestelle et al., 2001). These and other cell types such as skin (Hoeben et al., 1990; Fakharzadeh et al., 2000), endothelial cells (Dwarki et al., 1995; Chuah et al., 1995; Rosenberg et al., 2000), hepatocytes (Andrews et al., 1999), bone marrow stromal cells (Chuah et al., 1998) and hematopoietic cells (Hoeben et al., 1992; Evans & Morgan, 1998; Tonn et al., 2002) may be useful in gene therapeutic approaches. Indeed, the implantation of ex vivo modified fibroblasts that secreted FVIII was shown to be well tolerated and led to detectable FVIII plasma levels in patients with severe hemophilia A (Roth et al., 2001).

US patent application 2002/0042130 A1 and Lin et al. (2002) describe studies on the use of blood outgrowth endothelial cells (BOECs) for gene therapy for hemophilia A. Gehling et al. (2000) report the in vitro differentiation of endothelial cells from AC133-positive progenitor cells. Gehling et al. do not disclose, however, the transduction of cells with DNA encoding factor VIII. EP 1 136 553 A1 and WO 01/70968 A2 relate to the production of recombinant blood clotting factors in immortalized human cell lines.

Rosenberg et al. (2000) “Arteriosclerosis, thrombosis and vascular biology” 20, 2689-2695 describes the transduction of the B-domain deleted VIII gene in primary human umbilical vein endothelial cells (HUVECs). These cells coat the inner wall of the umbilical vein and thus represent fully differentiated endothelial cells which show very limited proliferative potential. Rosenberg et al. does not disclose the use of cells derived from cord blood, i.e. placental residual blood. The cells described by Rosenberg et al. differ from endothelial cells from cord blood in their phenotype and proliferative potential.

### SUMMARY OF THE INVENTION

It is an object of the present invention to provide cells that are suitable for producing sufficient amounts of a therapeutically effective protein. It is a further object of the invention to provide cells that are suitable for producing sufficient amounts of functional factor VIII protein and can be used in gene therapy.

It has surprisingly been found that endothelial precursor cells derived from human cord blood can be differentiated into mature endothelial cells that secrete high levels of factor VIII upon viral transduction with cDNA encoding factor VIII. The invention therefore relates to a method for the production of a protein, e.g., a blood coagulation factor, comprising culturing human endothelial cells derived from cord blood or bone marrow under suitable conditions; and isolating the desired protein from the cell culture medium.

### BRIEF DESCRIPTION OF THE DRAWINGS

FIG. 1. Cumulative growth curves of endothelial cells. In the representative experiments shown here, about 10⁶ CD34-positive cells from cord blood obtained from two pools of donors were cultured in EDM on gelatin-coated plates. The adherent cells were then passaged and cultured in EGM-2 until senescence. The graph defined by the black squares (WT; ▪) gives the number of endothelial cells plotted against the time after the first passage. On day 37 (A) and 23 (B), respectively, 10⁴ cells were plated for transduction 48 hours later with the construct cPPT-C(FVIIIΔB)IGWS (abbreviated as C(F8)IGWS; open circles; o) and pHR'SIN.cPPT-SEW (abbreviated as SEW; open triangles; Δ). These cells were also kept in EGM-2 until they ceased to proliferate.

FIG. 2. Morphology and phenotypic characterisation of CBECs (cord blood-derived endothelial cells). CBECs from four different batches were analyzed for uptake of DiI-Ac-LDL and expression of cell surface markers by flow cytometry at various time points during culture. The x-axis gives the fluorescence intensity of the analyzed cells. Cells that were labelled with DiI-Ac-LDL or antibodies are represented by the thick lines, whereas the thin lines represent the appropriate negative controls. The panels show that CBECs were uniformly positive for the uptake of DiI-Ac-LDL and for the endothelial markers VE-cadherin, CD146 and CD31. Subsets of cells expressed CD34 and, albeit very weakly, KDR. All CBECs were negative for CD133, HLA-DR, CD45 and CD14.

FIG. 3. Lentiviral transduction of endothelial cells. In four independent experiments, CBECs at passage numbers three to eight were transduced with the lentiviral constructs pHR'SIN.cPPT-SEW (abbreviated as SEW) and cPPT-C(FVIIIΔB)IGWS (abbreviated as C(F8)IGWS). (A) Transductions were performed with the constructs at MOIs of 10 and 100 for each vector, and transduction efficiencies were determined by analysis of EGFP expression with untransduced cells as negative controls. In (B), the histogram illustrates the level of EGFP expression detected by FACS. The diagram shows the result of a representative example of four experiments. FACS analysis was performed 30 days after transduction with C(F8)IGWS (MOI 10; black area under the curve) and with SEW (MOI 10; grey area under the curve). Untransduced cells served as negative controls (white area under the curve).

FIG. 4. Phenotype of transduced CBECs: Incorporation of DiI-Ac-LDL and in vitro tube formation. (A&B) Five weeks after transduction with pHR'SIN.cPPT-SEW (MOI 100), cells were incubated with DiI-Ac-LDL for one hour and examined for EGFP expression (A) and DiI-Ac-LDL uptake (B) by fluorescence microscopy. (C&D) For the tube formation assay, CBECs were detached by trypsin/EDTA treatment and plated on Matrigel® basement membrane matrix. The cells were incubated at 37° C. for 8 to 10 hours and examined microscopically for the formation of angiogenic tubes (C: phase contrast; D: EGFP expression).

FIG. 5. Quantification of FVIII:C in CBEC supernatants. CBECs from three pools of donors were transduced with the BDD FVIII-encoding construct cPPT-C(FVIIIΔB)IGWS at the MOI of 10. In order to quantify secretion of FVIII:C at various timepoints after transduction, 5×10⁴ cells were plated in 1 ml of EGM-2 and incubated at 37° C. After 48 hours, supernatant was harvested, cleared of cellular debris by short centrifugation and stored at −80° C. until analysis. Every assay included supernatans from cells transduced with pHR'SIN.cPPT-SEW and from untransduced cells. No FVIII:C could be detected in any of these controls (detection limit 0.01 IU/ml).

FIG. 6. Detection of FVIII protein in concentrated CBEC supernatants by immunoblotting. Serum-free culture supernatants from untransduced CBECs and from CBECs that were transduced with cPPT-C(FVIIIΔB)IGWS were concentrated 200 to 400fold by ultrafiltration. The samples were separated by SDS-PAGE and subjected to immunoblotting. Lanes 1 and 2 show commercially available FVIII concentrates as reference. Lanes 3 and 4 contain concentrated supernatant from CBECs. In order to detect signals of various intensities, the autoradiography film was exposed for different periods of time (1a-4a: one minute; 1b-4b: 20 minutes). Lane 1: Oct, Octanate® (plasma-derived full-length FVIII); lane 2: ReF, ReFacto® (recombinant BDD FVIII); lane 3: supernatant from cPPT-C(FVIIIΔB)IGWS transduced CBECs (BDD FVIII); lane 4: supernatant from untransduced CBECs.

FIG. 7 shows the amino acid sequence of human factor VIII (amino acids 20-2351 correspond to mature factor VIII).

### DESCRIPTION OF EXEMPLARY EMBODIMENTS

The cells that are used in the method according to the invention are obtainable by a process comprising the steps a) contacting in vitro human cord blood-derived endothelial precursor cells and/or endothelial precursor cells obtained from bone marrow with at least one growth factor, whereby the growth factor(s) promote(s) the differentiation of endothelial precursor cells into mature endothelial cells; and b) transducing the endothelial precursor cells or the mature endothelial cells with DNA encoding the protein. The order of steps a) and b) can be changed.

The term “growth factor” refers to a substance, e.g., a protein, that is capable of inducing proliferation and/or differentiation of mammalian cells.

A “population of cells” designates a composition comprising at least two cells. The population generally comprises at least 100 cells, preferably at least 1000 cells. The cells may be homogeneous in respect to expression of a given marker protein, they may also be heterogeneous with respect to expression of another marker protein.

As used herein, the term “endothelial cell” or “mature endothelial cell” designates a cell that expresses the markers VE-cadherin (CD144), CD146, CD31 and LDL-receptor. “Endothelial cells” or “mature endothelial cells” may still have significant proliferative potential greater than that of fully differentiated endothelial cells.

A “differentiated endothelial cell” or “fully differentiated endothelial cell” refers to an endothelial cell that has undergone terminal differentiation. Differentiated endothelial cells thus have very limited proliferative potential.

An “endothelial precursor cell” (EPC) is a cell that can be differentiated into a mature endothelial cell. An EPC usually expresses CD34, AC133 and/or fibroblast growth factor 1 receptor (FGF1-R) and is characterized by its proliferative potential.

In a “population of endothelial cells” at least 10% of the cells are endothelial cells, preferably at least 25%, more preferably at least 50%, most preferably at least 75%.

As used herein, the term “CD34-positive cells” denotes cells that express the surface marker protein CD34. Expression of CD34 can be determined by immunofluorescence analysis or FACS analysis using an antibody directed against CD34.

A “blood coagulation factor” is a substance, e.g. a protein, which has the coagulant activity of a component of the intrinsic pathway of the blood coagulation cascade. Components of the blood coagulation cascade include, but are not limited to, factor V, factor VII, factor VIII, factor IX, VWF, and the like. “Factor VIII” denotes a substance, e.g. a protein, that has the ability, when administered to patients with Hemophilia A, to correct the clotting defect. As non-limiting examples, this definition includes full length recombinant factor VIII and B domain deleted factor VIII. “Factor IX” denotes a substance, e.g. a protein, that has the ability, when administered to patients with Hemophilia B, to correct the clotting defect.

The term “transducing” refers to a process wherein DNA is transferred into a cell. Viral or non-viral vectors may be used in this process. The cells are preferably human cells. The DNA may or may not integrate into the genome of the cell. For transduction, vectors may be used which contain a sequence encoding a polypeptide to be expressed. A promoter is usually operably linked to the coding sequence, i.e. the promoter is located within the vector in a manner that it can stimulate transcription of the DNA sequence encoding the protein, e.g. the blood coagulation factor.

The term “recombinant DNA” denotes a DNA molecule that has been prepared by joining DNA fragments from different sources. Such recombinant DNA molecules include cDNA and genomic clones. Recombinant DNA has been made by human intervention. Recombinant DNA molecules in accordance with the present invention are in a form suitable for use within genetically engineered protein production systems. The DNA may include naturally occurring 5′- and 3′-untranslated regions such as promoters and terminators.

Preferably, the human endothelial precursor cells are derived from cord blood. The endothelial precursor cells may express at least one surface marker selected from the group consisting of CD34, AC133, CD146 and FGF1-R. In a particular embodiment the endothelial precursor cells express at least two, alternatively at least three, alternatively all of the said four cell surface marker proteins. Usually at least 10% of the population of endothelial precursor cells are positive for one of the above mentioned cell surface markers, preferably at least 25%, more preferably at least 50%, even more preferably at least 75%, most preferably at least 80%. The endothelial precursor cells may be obtained by enriching cells that are positive for one of the above mentioned cell surface markers. In a first step, mononuclear cells may be isolated from cord blood by Ficoll density separation. After washing of the mononuclear cells CD34-positive cells or AC133-postive cells or CD146-positive cells or FGF1-R-positive cells can be enriched using magnetic activated cell sorting. After a first step of enrichment, a second step of enrichment may be performed using another cell surface marker protein as target.

The human endothelial precursor cells can also be obtained from bone marrow. Bone marrow cells may be obtained by aspiration, and suitable endothelial precursor cells may be selected and amplified by suitable methods as described herein. Alternatively, human endothelial precursor cells obtained from cord blood and bone marrow may be mixed.

When CD34-positive cells are to be contacted with at least one growth factor, these cells may be obtained by enriching CD34-positive cells from human cord blood. Suitable enrichment techniques are known to those skilled in the art. In a first step, mononuclear cells may be isolated from blood by Ficoll density separation. After washing of the mononuclear cells, CD34-positive cells can be enriched using magnetic activated cell sorting. Using this method, the fraction of CD34-positive cells can be enriched from less than 2% in the mononuclear cell fraction to more than 80% in the enriched fraction. Isolation of CD34-positive cells by fluorescence-activated cell sorting (FACS) may also be contemplated.

In the CD34-positive cells that are contacted with at least one growth factor usually at least 10% of the cells are CD34-positive, preferably at least 25%, more preferably at least 50%, even more preferably at least 75%, most preferably at least 80%. In a preferred embodiment, the purity of CD34-positive cells is in the range of 80% to 95%. Populations of cells with purities of CD34-positive cells in this range can be obtained by enriching the cells as described above.

The cells may be cultured in an appropriate medium in tissue-culture plates or flasks prior to contacting them with the growth factor(s) for differentiation into endothelial cells.

According to the method of the invention, the cord blood-derived endothelial precursor cells are contacted in vitro with at least one growth factor, wherein the at least one growth factor promotes the differentiation of endothelial precursor cells into mature endothelial cells. Whether a growth factor promotes the differentiation of endothelial precursor cells into mature endothelial cells can be determined by contacting CD34-positive cells derived from cord blood with a growth factor and determining whether one or more of the endothelial markers CD144, CD146, CD31 and LDL-receptor are expressed.

In one embodiment, the cells are contacted with vascular endothelial growth factor (VEGF). In another embodiment, VEGF may be combined with another growth factor, e.g., basic fibroblast growth factor (bFGF), stem cell factor (SCF) or stem cell growth factor-β (SCGF-β). The endothelial precursor cells may be contacted with one of the following combinations of growth factors: VEGF+bFGF; VEGF+SCF; VEGF+SCGF-β; VEGF+bFGF+SCF; VEGF+bFGF+SCGF-β; VEGF+SCF+SCGF-β. Most preferably, the endothelial precursor cells are contacted with the four growth factors VEGF, bFGF, SCF and SCGF-β. Usually, the growth factors are added to the cell culture medium. Preferably, the growth factors are human growth factors. The growth factors may be produced by recombinant expression.

The amino acid sequences and cDNA sequences of human VEGF, human basic FGF, human SCF and human SCGF-β are known (VEGF: Conn et al., Proc Natl Acad Sci, USA 1990 April 87(7):2628-32; Tischer E. et al., J Biol Chem 1991 June 25, 266(18):11947-54; bFGF: Kurokawa T. et al., FEBS Lett. 1987 March 9, 213(1):189-94; SCF: Martin F. H. et al., Cell 1990 October 5, 63(1):203-11; SCGF-β: Hiraoka A. et al., Proc Natl Acad Sci, USA 1997 July 8, 94(14):7577-82; Bannwarth S. et al., J Biol Chem 1998 January 23, 273(4):1911-6.

VEGF can be administered to the cell culture medium of the EPCs at a concentration of 1 to 1000 ng/ml, preferably 10 to 200 ng/ml, more preferably 25 to 100 ng/ml, most preferably about 50 ng/ml. Basic FGF is usually added to the medium at a concentration of 1 to 500 ng/ml, preferably 5 to 100 ng/ml, more preferably 10 to 50 ng/ml, most preferably about 20 ng/ml. SCF may be added at a concentration of 1 to 1000 ng/ml, preferably 10 to 200 ng/ml, more preferably 25 to 100 ng/ml, most preferably about 50 ng/ml. SCGF-β can be added to the medium at a concentration of 1 to 500 ng/ml, preferably 5 to 100 ng/ml, more preferably 10 to 50 ng/ml, most preferably about 20 ng/ml.

The growth factors may be added to the cells simultaneously or successively. The order of addition can be varied. It is preferred, however, that the growth factors are added to the cells simultaneously.

In one embodiment of the invention, at least one additional growth factor is added to the cells when inducing the differentiation into an endothelial phenotype. Optionally, at least two, at least three, or at least four additional growth factors are added to the cells for the induction of the endothelial phenotype. Examples of additional growth factors include but are not limited to insulin-like growth factor (IGF), epidermal growth factor (EGF), and the like.

In another embodiment, at least one inhibitor of 3-hydroxy-3-methylglutaryl-coenzyme A (HMG-CoA) such as Atorvastatin is added to the cells for the induction of the endothelial phenotype. The HMG-CoA inhibitor (e.g. Atorvastatin) may be added in combination with one or more growth factors or cytokines.

The cells can be cultured according to methods known in the art. They may be grown in plates or flasks coated with gelatin or collagen at a density of e.g. 5×10⁴ to 1×10⁵ cells/cm². The EPCs differentiate into (mature) endothelial cells due to the presence of the growth factors.

Usually, at least 10%, preferably at least 25%, more preferably at least 50%, even more preferably at least 75%, most preferably at least 90% of the cells in the population of endothelial cells express the marker protein CD144. Usually, at least 10%, preferably at least 25%, more preferably at least 50%, even more preferably at least 75%, most preferably at least 90% of the cells in the population of endothelial cells express the marker protein CD146. Usually, at least 10%, preferably at least 25%, more preferably at least 50%, even more preferably at least 75%, most preferably at least 90% of the cells in the population of endothelial cells express the marker protein CD31. Usually, at least 10%, preferably at least 25%, more preferably at least 50%, even more preferably at least 75%, most preferably at least 90% of the cells in the population of endothelial cells express the marker protein LDL-receptor. The endothelial cells in the population of cells preferably are capable of forming tubes in a Matrigel assay as described in the examples. In a preferred embodiment, the population of endothelial cells is characterized by a substantially uniform expression of the marker molecules CD144 (VE-cadherin), CD146, CD31 and/or LDL-receptor. More preferably, two, three, or all of CD144, CD 146, CD31 and LDL-receptor are expressed in a substantially uniform manner. The endothelial cells of the invention are further characterized by expression of VWF, which is secreted. When the cells express factor VIII, the molar ratio of VWF:FVIII in the supernatant can vary between 1:1 and 100:1. Furthermore, the cells may be capable of binding Ulex europaeus agglutinin. Usually, at least 10%, preferably at least 25%, more preferably at least 50%, even more preferably at least 75%, most preferably at least 90% of the cells in the population of endothelial cells express VWF. Usually, at least 10%, preferably at least 25%, more preferably at least 50%, even more preferably at least 75%, most preferably at least 90% of the cells in the population of endothelial cells are capable of binding Ulex europaeus agglutinin.

The endothelial cells of the invention preferably do not express the marker molecules CD45, CD14, CD133 and/or HLA-DR. Most preferably, none of the four marker molecules is expressed in the cells according to the invention. A fraction of the cells may express the marker CD34, e.g. 1 to 90%, preferably 5 to 50% of the cells.

In a specific embodiment, less than 90% of the cells express KDR (=VEGF receptor-2=flk-1). Alternatively, less than 50% or less than 25% or less than 10% of the cells express KDR. In another embodiment, more than 90% of the cells express KDR.

The various embodiments concerning the expression of markers as described supra may be combined.

In a further step the endothelial cells are transduced with DNA encoding a therapeutically effective protein, e.g. a blood coagulation factor. Examples of therapeutically effective proteins include but are not limited to 1. blood coagulation factors, 2. growth factors (e.g. NEGF, FGF, SCF), 3. chemokines. The DNA may encode blood coagulation factor VIII, IX, or the like. Preferably, the DNA encodes human factor VIII. The DNA may code for mature wild type factor VIII or a mutein thereof. The factor VIII mutein may be a mutein having point mutations, a mutein being truncated at its C- or N-terminus, and/or a mutein partially or entirely lacking its B-domain.

U.S. Pat. No. 6,346,513, WO 86/06101, WO 92/16557, and EP 0 123 945 describe deletions of the sequence coding for the B domain of factor VIII. EP 1 233 064, US 2002/0165177 and U.S. Pat. No. 6,271,025 describe the insertion of introns into the cDNA encoding factor VIII. U.S. Pat. No. 5,422,260 describes point mutations in the DNA sequence encoding factor VIII. The modifications in the coding or non-coding DNA sequence described therein can be used in accordance with the present invention. Also combinations of the modifications described may be employed.

U.S. Pat. No. 6,228,620, U.S. Pat. No. 5,789,203 and U.S. Pat. No. 5,693,499 describe the coexpression of DNA coding for the heavy chain and DNA coding for the light chain of factor VIII. These embodiments may also be used in accordance with the present invention.

In a particular embodiment, the factor VIII mutein has at least one of the following mutations (EP 1 136 553 A1):
valine at position 162 is replaced by another neutral amino acid residue;
serine at position 2011 is replaced by another hydrophilic amino acid residue;
valine at position 2223 is replaced by an acidic amino acid residue;
the B-domain between positions arginine 740 and glutamic acid 1640 is replaced by an arginine-rich linker peptide comprising 10 to 25, preferably 14 to 20 amino acid residues.

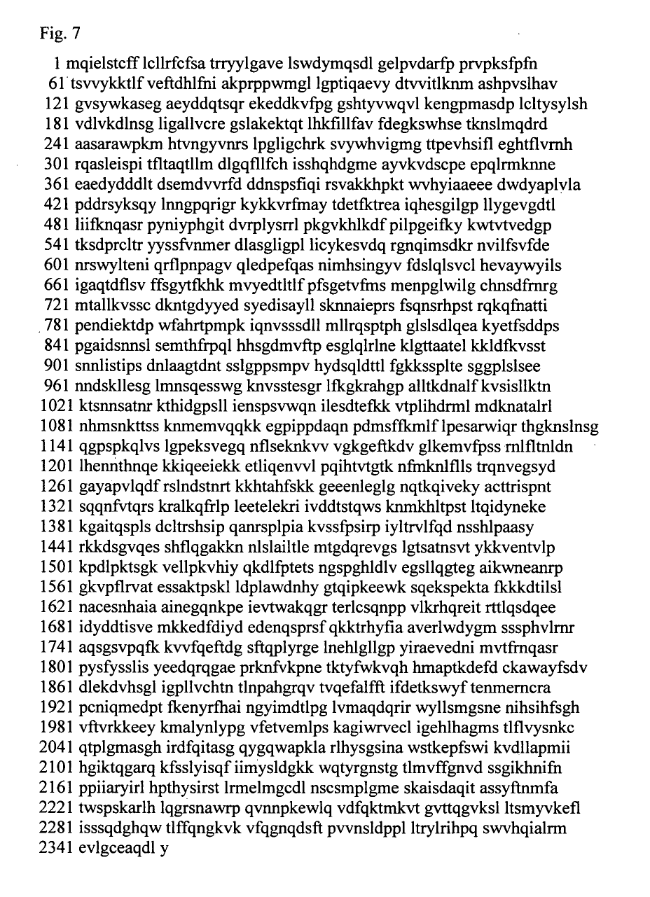

The positions refer to the published amino acid sequence of mature human factor VIII (Toole et al., Nature 1984, 312(5992):342-7; Wood et al., Nature 1984, 312(5992):330-7; Gitschier et al., Nature 1984, 312(5992):326-30). The amino acid sequence of human factor VIII is shown in FIG. 7 (SEQ ID NO:3). In another embodiment, the DNA encoding factor VIII is a modified factor VIII cDNA, wherein at least one intron has been inserted into at least one location of factor VIII cDNA.

The DNA encoding factor VIII is usually part of a vector that is used for transducing cells. The vector may comprise a promoter operably linked to the DNA sequence coding for the blood coagulation factor. It is preferred that the vector is a viral vector, preferably a retroviral vector, more preferably a lentiviral vector. More preferably, the lentiviral vector is an HIV-1-based vector. Besides HIV-1, lentiviral vectors based on HIV-2, simian immunodeficiency virus, equine infectious anemia virus, feline immunodeficiency virus (FIV) and visna virus may be used. In another embodiment the FVM encoding vector may also be a plasmid DNA (e.g. pcDNA3).

The endothelial cells of the invention can be cryopreserved and then be thawed and returned to culture without significant loss of their capacity to proliferate. To cryopreserve the cells, the culture cells can be detached and resuspended in suitable cryopreservation medium, i.e., containing a cryopreservation agent such as sugar(s), BSA, dimethyl sulfoxide (DMSO), glycerol, glycerol esters and the like.

In accordance with the present invention the cells may be frozen and thawed prior to transduction with DNA or after transduction with DNA. In another aspect of the invention, the cells are transduced prior to differentiating them from EPCs into mature endothelial cells.

In one embodiment, the transduced endothelial cells may be expanded by 4 to 10 orders of magnitude (10⁴ to 10¹⁰-fold), preferably by 5 to 9 orders of magnitude (10⁵ to 10⁹-fold). This expansion or proliferation step may be carried out after completion of steps a) and b). The expansion step is preferably carried out in vitro.

The endothelial cells described above are useful in a method for the production of a blood coagulation factor. The method comprises culturing endothelial cells according to the invention under suitable conditions; and isolating the blood coagulation factor from cell culture medium. The isolation step may comprise purifying the blood coagulation factor from the medium. Suitable purification steps include, but are not limited to, immunoaffinity chromatography, anion exchange chromatography, etc., and combinations thereof. Detailed purification protocols for coagulation factors from human blood plasma are, e.g., disclosed in WO 93/15105, EP 0 813 597, WO 96/40883 and WO 96/15140/50. They can be adapted to the requirements needed to isolate recombinant factors VIII and IX. For factor IX an effective protocol has been introduced containing an ammonium sulfate precipitation step followed by DEAE and HIC tentacle chromatography as well as heparin affinity chromatography (U.S. Pat. No. 5,919,909). Quantity and activity of the purified protein during and after the purification procedure may be monitored by ELISA and coagulation assays.

Preferably, the blood coagulation factor is human factor VIII. In this case, factor VIII may be purified in the presence of VWF. The VWF may be present in the cell culture medium and is preferably used in an amount of 1 to 100, more preferably 50 to 60 mol VWF per mol factor VIII.

In case of the production of factor IX, the culturing is preferably performed in the presence of vitamin K which may be present in an amount of 0.1 to 100 μg/ml culture broth, more preferably 1 to 20 μg/ml culture broth.

A composition obtained by the method for the production of a protein, e.g., a blood coagulation factor can be subjected to virus inactivation treatment. A virus inactivation treatment includes heat treatment (dry or in liquid state, with or without the addition of chemical substances including protease inhibitors). After virus inactivation a further purifying step for removing the chemical substances may be necessary. In particular, for factor VIII isolated from blood plasma the recovery of a high purity virus-inactivated protein by anion exchange chromatography was described (WO 93/15105). In addition several processes for the production of high-purity, non-infectious coagulation factors from blood plasma or other biological sources have been reported. Lipid coated viruses are effectively inactivated by treating the potentially infectious material with a hydrophobic phase forming a two-phase system, from which the water-insoluble part is subsequently removed. A further advantage has been proven to complement the hydrophobic phase treatment simultaneously or sequentially with a treatment with non-ionic biocompatible detergents and dialkyl or trialkyl phosphates (WO 96/36369, EP 0 131 740, U.S. Pat. No. 6,007,979). Non-lipid coated viruses require inactivation protocols consisting in treatment with non-ionic detergents followed by a heating step (60-65° C.) for several hours (WO 94/17834).

A pharmaceutical composition comprising an effective amount of the isolated protein, e.g., the coagulation factor may further comprise pharmaceutically acceptable additives including human serum albumin (HSA; preferably about 1 mg/ml solution); inorganic salts such as CaCl₂ (preferably 2 to 5 mM), amino acids such as glycine, lysine, and histidine (preferably 0.1 to 1 M per amino acid); disaccharides such as sucrose and/or trehalose (preferably 0.4 to 1 M); organic salts such as Na-citrate (preferably up to 50 mM); etc. The preparations may be aqueous or non-aqueous. In the latter case the major component is glycerol and/or polyethylene glycol (e.g., PEG-300). The preparation may also be in the dry form (to be dissolved in the desired solvent prior to administration).

Another aspect of the invention is the use of a population of cells according to the invention expressing factor VIII or IX for the preparation of a medicament for the treatment of hemophilia A or B, preferably hemophilia A. The cells of the invention expressing factor VIII can be used in an ex vivo gene therapy protocol for hemophilia A. The transduced endothelial cells according to the invention expressing factor VIII or IX may be transferred into an individual suffering from hemophilia A or B. Transfer methods include, but are not limited to the transplantation of synthetic vessels or prosthetic valves lined with transduced cells or the transplantation of a device or matrix designed to house transduced endothelial cells. The cells may also be introduced into the blood stream of a patient by conventional means such as intravenous infusion over a period of time.

Yet another aspect of the invention is the use of a population of cells according to the invention for the preparation of a medicament for the treatment of disorders of the cardiovascular system. These disorders include, but are not limited to, cardiac failure, acute and chronic heart disease. According to this aspect, the cells preferably provide a depot of autologous cells for the ectopic synthesis and secretion of any protein with systemic effect via the circulation. A particular aspect is revascularization in acute and chronic heart disease.

Usually, the cells according to the invention show a better therapeutic effect, since they may integrate into affected tissue in an improved manner. Also the homing of these cells may be significantly improved.

The cells according to the invention can be expanded by 4 to 10 orders of magnitude, preferably by 5 to 9 orders of magnitude and have the capacity to secrete a protein, e.g. factor VIII, upon lentiviral transduction. Therefore, endothelial cells prepared in accordance with the present invention can be used in hemophilia A gene therapy. In a specific embodiment, the expansion of the cells is limited. Accordingly, the endothelial cells may cease to proliferate within 15 weeks, preferably within 12 weeks, optionally within 8 or 9 weeks, starting from the addition of at least one growth factor for differentiation into endothelial cells. Cells with limited proliferation potential are preferred for use in a gene therapy protocol.

Endothelial cells prepared in accordance with the present invention express high levels of factor VIII. The amount of factor VIII secreted by the cells is at least 2.5 IU/10⁶ cell/48 h, preferably at least 5 IU/10⁶ cell/48 h, most preferably at least 7 IU/10⁶ cell/48 h.

According to another aspect, the invention relates to a method for preparing an immortalized cell line from the population of endothelial cells described herein. The method includes transformation of the endothelial cells. The cells may be converted to an immortalized cell line by viral and/or non-viral infection (e.g. human papilloma virus, Epstein-Barr virus, DNA-plasmids), transfer of genes coding for human telomerase submit proteins such as telomerase reverse transcriptase (hTERT) or of one or more oncogenes. This embodiment is preferred for in vitro-production and subsequent isolation of blood coagulation factor, e.g., FVIII. The immortalized cell line is within the scope of the present invention.

### EXAMPLES

The following non-limiting examples further illustrate the invention.

### 1. Materials & Methods

Isolation of CD34-Positive Cells from Cord Blood

Mononuclear cells were isolated by Ficoll density separation (d=1.077 g/ml) from cord blood from healthy donors and contained less than 2% CD34⁺ cells as determined by immunofluorescence staining and fluorescence-activated cells sorting (FACS) analysis. The cells were washed twice in Dulbecco's PBS (BioWhittaker, Verviers, Belgium) containing 2 mM EDTA (Sigma, Taufkirchen, Germany) and 0.5% human serum albumin (HSA; DRK-Blutspendedienst Niedersachsen, Springe, Germany), and CD34-positive cells were enriched by magnetic activated cell sorting (MACS) using the Direct CD34 Progenitor Cell Isolation Kit (Miltenyi Biotec, Bergisch Gladbach, Germany) according to the manufacturer's instructions. The purity of CD34⁺ cells was in the range of 80% to 95%.

Differentiation of Endothelial Cells

CD34⁺ cells were resuspended in endothelial differentiation medium consisting of 80% basal Iscove medium (Biochrom, Berlin, Germany), 10% horse serum (PAN Biotech, Aidenbach, Germany; selected lots), 10% heat-inactived fetal calf serum (FCS; Biochrom, selected lots), L-alanyl-L-glutamine (final concentration 2 mM; BioWhittaker) and penicillin-streptomycin (final concentrations 100 U/ml and 100 μg/ml, respectively; BioWhittaker). The medium was filtered (pore size 0.22 μm) and then supplemented with recombinant human (rh) VEGF (vascular endothelial growth factor; 50 ng/ml), rh basic FGF (fibroblast growth factor-2; 20 ng/ml), rh SCF (stem cell factor; 50 ng/ml; all from R&D Systems, Wiesbaden, Germany), and rh SCGF-β (stem cell growth factor-β; 20 ng/ml; Pepro Tech, Frankfurt am Main, Germany). This medium including the growth factors will be referred to as endothelial differentiation medium (EDM).

CD34⁺ cells were cultured in tissue-culture treated 6-well plates coated with 1% gelatin solution (Sigma) at 5×10^{4-1×10}⁵ cells/cm² in 3 ml per well at 37° C. with 5% CO₂ in a humidified atmosphere. If the cell yield from one donor was lower than 5×10⁵, the cells were cultured in 24-well plates, or cells from different donors were pooled. Half of the medium was exchanged twice a week, and the cells were incubated until the wells were 80%-90% confluent with adherent cells (usually up to three weeks).

Adherent cells were detached by trypsin/EDTA treatment and then cultured on gelatin-coated tissue culture flasks in medium EGM-2 (endothelial cell basal medium (EBM)-2 supplemented with 2% FCS, VEGF, rh EGF (epidermal growth factor), rh basic FGF, R³-IGF-1 (insulin-like growth factor), hydrocortisone, ascorbic acid, heparin, gentamicin, and amphotericin B; Clonetics/BioWhittaker, Verviers, Belgium). Cells could be expanded most efficiently when grown at low density (1×10³/cm²).

Immunofluorescence

Approximately 10⁵ cells were prepared for flow cytometry by washing with PBS containing 1% FCS (Biochrom) and 0.1% sodium azide (Sigma). In order to characterize the expression of hematopoietic and endothelial cell surface markers, the following monoclonal antibodies (mAb) were used: anti-CD14-PE, anti-CD34-PE, anti-CD45-FITC, anti-HLA-DR-FITC (all from BD Biosciences, Heidelberg, Germany), anti-CD133-PE (Miltenyi Biotec), anti-CD146, anti-CD146-FITC, anti-VE-cadherin (all from Chemicon, Temecula, Calif.), anti-34-PC5 (Immunotech, Marseille, France), anti-CD31-FITC and anti-KDR (both from Sigma). Cells were incubated with the respective mAb for 20 minutes at 20° C. (anti-CD31-FITC, anti-KDR) or at 4° C. (all others). In a second step, unconjugated primary mAb were detected by incubation with RPE-conjugated F(ab′)₂ fragment of rabbit anti-mouse immunoglobulin (Dako, Glostrup, Denmark). Samples of cells were also stained with isotype-matched control antibodies (purchased from BD Biosciences and Immunotech). FACS analyses were performed using a FACScan flow cytometre (BD Biosciences) and Cell Quest software (BD Biosciences). Each analysis included at least 10,000 events. Dead cells were excluded based on their forward scatter and side scatter properties.

DiI-Ac-LDL Labeling

Fluorescent labeling of endothelial cells by uptake of acetylated low-density lipoprotein (Ac-LDL) was performed by incubating the cells with 2 μg/ml DiI-Ac-LDL (Harbor Bio-Products, Norwood, Mass.) in EGM-2 for 60 minutes at 37° C. Then the medium was exchanged for EGM-2, and the DiI-Ac-LDL uptake of the cells was analyzed by fluorescence microscopy (Nikon Eclipse TE300) and flow cytometry.

Lentiviral Constructs

Two HIV-1-derived self-inactivating lentiviral gene transfer constructs were used in this example: The vector pHR'SIN.cPPT-SEW (Demaison et al., 2002) contains a gene expression cassette consisting of the enhancer region of the spleen focus forming virus (U3-LTR), the cDNA of the enhanced green fluorescent protein (EGFP) and the Woodchuck hepatitis virus posttranscriptional regulatory element (WPRE).

In order to generate the vector cPPT-C(FVIIIΔB)IGWS, three cloning steps were required. First, the HIV-1-based monocistronic self-inactivating vector pRRL-CMV-GFP-WPRE-SIN (CGWS, derivative of the vectors pRRL-PGK-GFP-SIN-18 and pHR'-CMV-lacZ-SIN-18 kindly provided by R. Zufferey, Geneva, Switzerland) encoding the EGFP gene as a marker gene flanked 5′ by an internal CMV promoter and 3′ by the WPRE sequence was modified by insertion of a multiple cloning site (MCS) and the internal ribosome entry site (IRES) of the encephalomyocarditis virus (ECMV; MCS and IRES from pIRES2-EGFP, Clontech, Heidelberg, Germany). In a second step, the central polypurine tract and central termination sequence (cPPT/CTS; Charneau et al., 1992; Zennou et al., 2000; Sirven et al., 2000; Follenzi et al., 2000) from HIV-1 was cloned into the ClaI site 5′ of the internal CMV promoter. The cPPT/CTS fragment flanked by ClaI restriction sites was obtained by polymerase chain reaction (PCR) using the oligonucleotides PPTCTSCLA-5: 5′-CCA TCG ATA CAA ATG GCA TTC ATC C-3′ (SEQ ID NO:1) and PPTCTSCLA-3: 5′-CCA TCG ATC TCG AGC CAA AGT GGA TCT CTG CTG TCC-3′ (SEQ ID NO:2) with a plasmid containing the HIV-1 LAI gag-pol cDNA (Myers et al., 1989) as template. Finally, the cDNA for human B-domain deleted FVIII (huBDD FVIII; deletion as described in Tonn et al., 2002) was cloned into the SalI site of the MCS between CMV promoter and IRES to yield the bicistronic vector cPPT-C(FVIIIΔB)IGWS.

Production of Lentiviral Supernatants

To generate lentiviral particles, gene transfer vector DNA was transiently introduced into 293T cells (cultured as decribed in Tonn et al., 2002) by triple co-transfection with the packaging construct pCMVΔR8.91 (Zufferey et al., 1997) encoding gag, pol, rev, and tat and the pseudotyping construct pMD2.VSVG (Follenzi and Naldini, 2002) coding for the vesicular stomatitis virus glycoprotein (VSV-G).

Transfection of plasmid DNA was performed by calcium phosphate coprecipitation. Sixteen hours after transfection, cells were given fresh medium (DMEM [Gibco, Karlsruhe, Germany] supplemented with 10% heat-inactivated FCS, 4 mM L-glutamine, and penicillin-streptomycin at 100 U/ml and 100 μg/ml, respectively). After further 24 hours, the viral supernatant was collected, filtered (pore size 0.22 μm), concentrated by ultracentrifugation and filtered again. Virus titers were determined as 293T-transducing units (TU/ml) by transduction of 293T cells with dilutions of vector concentrate and subsequent FACS analysis. Concentrated vector stocks of the constructs pHR'SIN.cPPT-SEW and cPPT-C(FVIIIΔB)IGWS had titers between 10⁸ and 10⁹ and between 10⁷ and 10⁸ TU/ml, respectively.

Transduction of Endothelial Cells

Endothelial cells were plated on gelatin-coated 6-well plates at 1×10⁴ cells per well in EGM-2 and incubated at 37° C. for 48 hours. For the transduction, the medium was exchanged for unconcentrated or concentrated virus supernatant in EGM-2 in the presence of 4 μg/ml protamine sulfate (Sigma) and 50 μM dNTPs (New England Biolabs, Frankfurt am Main, Germany). After spinoculation (1250 g, 90 minutes, 32° C.), the cells were incubated for further 16 hours at 37° C. Then the virus supernatant was removed and the cells were given fresh EGM-2. The multiplicities of infection (MOIs) were calculated as ratios of 293T-TU/ml to target cells. EGFP expression was analyzed by FACS analysis and fluorescence microscopy at various time points.

FVIII Quantification

To assess the amount of secreted FVIII, transduced endothelial cells and untransduced control cells were seeded at 5×10⁴ cells in a total volume of 1 ml EGM-2 in gelatin-coated 12-well plates. After 48 hours, the supernatants were cleared of cellular debris by centrifugation (700 g, 3 minutes, 4° C.) and stored in multiple aliquots at −80° C. until analysis. FVIII antigen (FVIII:Ag) and FVIII activity (FVIII:C) were determined with the commercially available Immunozym FVIII:Ag ELISA and the Immunochrom FVIII:C chromogenic assay respectively (Immuno, Heidelberg, Germany) according to the manufacturer's instructions. FVIII levels are given as international units (IU)/ml with 150 ng/ml corresponding to 1.0 IU/ml. FVIII standards in both assays were calibrated against WHO plasma standards by the manufacturer.

FVIII Western Blot

Confluent layers of endothelial cells were washed with PBS and cultured with EGM-2 without FCS for 15-24 hours. Culture supernatants were filtered (0.22 μm) to remove cellular debris and concentrated up to 400fold by ultracentrifugation in Vivaspin 20 concentrators (Sartorius, Gottingen, Germany) with a molecular weight cut-off (MWCO) of 30 kDa. The concentrates were boiled for 5 minutes in Laemmli buffer (RotiLoad 1; Roth, Karlsruhe, Germany), separated by SDS-PAGE on 10% polyacrylamide gels and transferred to a polyvinylidine difluoride membrane (Roth). The membranes were blocked for 3 hours at 20° C. in 5% powdered skim milk and incubated at 4° C. for 15-20 hours with polyclonal sheep anti-human factor VIII:C antibody (Enzyme Research Laboratories, Swansea, UK). After extensive washing, the blots were incubated with a peroxidase-conjugated donkey anti-sheep IgG secondary antibody (Sigma) for 40 minutes at room temperature. After further washing, the proteins were visualized by enhanced chemiluminescence (Pierce, Bonn, Germany). As positive controls, we used recombinant B-domain deleted FVIII ReFacto® (Pharmacia & Upjohn, Martinsried, Germany) and plasma-derived human FVIII (Octanate®; kindly provided by Lothar Biesert, Octapharma, Frankfurt am Main, Germany).

In Vitro Matrigel Assay

Prechilled 24-well plates were coated with 500 μl Matrigel® basement membrane matrix (BD Biosciences) per well and incubated for 1 hour at 37° C. Endothelial cells were harvested by trypsin/EDTA treatment, resuspended in EGM-2, and seeded on top of the gelled Matrigel® at 6×10⁴ to 1×10⁵ cells in 400 μl. Cultures were incubated at 37° C. After 8 to 10 hours, the cultures were checked for tube formation by phase contrast and fluorescence microscopy (Nikon Eclipse TE300).

Statistical Analysis

Data are presented as means±SD. The paired Student's t-test was used to compare transduction efficiencies and FVIII secretion levels of cells transduced at different multiplicities of infection (MOIs). Statistical analysis was performed using the GraphPad Prism 3.0 software.

### 2. Results

Differentiation and Culture of Cord Blood-Derived Endothelial Cells (CBECs)

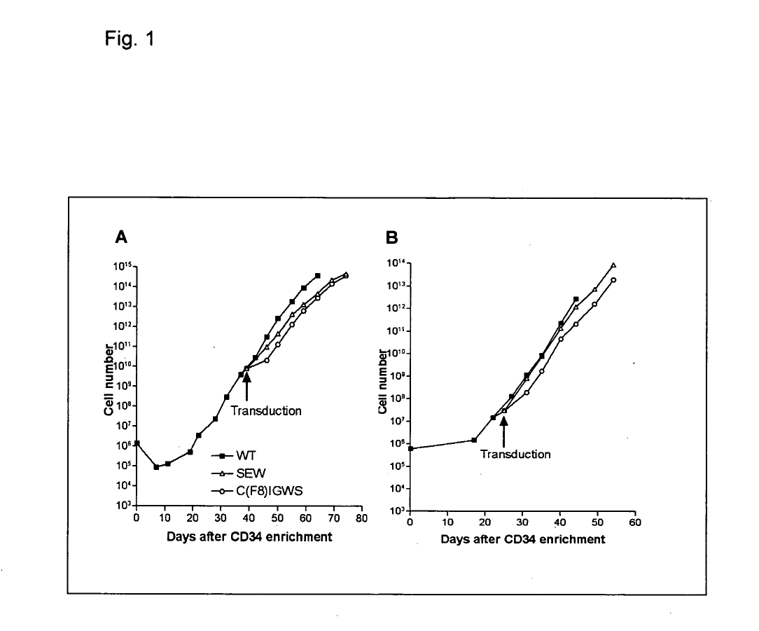

After isolation of cord blood mononuclear cells, CD34⁺ cell fractions with a purity of 80% to 95% were obtained using MACS immunomagnetic beads. Cells from single donors or cell pools from several donors were cultured in EDM containing rh VEGF, rh basic FGF, rh SCF and rh SCGF-β (see Materials & Methods) for about three weeks. When proliferating adherent cells with endothelial morphology could be detected, they were passaged before reaching confluency. With an input of 10⁵ to 10⁷ CD34⁺ cells, adherent cells could be expanded up to 10⁹ fold during a total culture time of eight weeks. FIG. 1 shows the cumultative growth curves of two representative experiments with cells from donor pools (denoted as ‘WT’). In this example, cells proliferated with a doubling time of about 30-35 hours and were expanded up to more than 108 fold before they ceased to grow and the culture became senescent.

Cultures with mononuclear cells from CB prior to CD34 isolation and with the CD34-depleted fraction either did not yield any detectable EC differentiation under otherwise identical conditions or yielded a considerably lower cell number. Therefore, the enrichment of CD34-expressing cells is preferred. The data suggest that the EC were derived from CD34⁺ progenitor cells.

Phenotypic Characterisation of CBECs

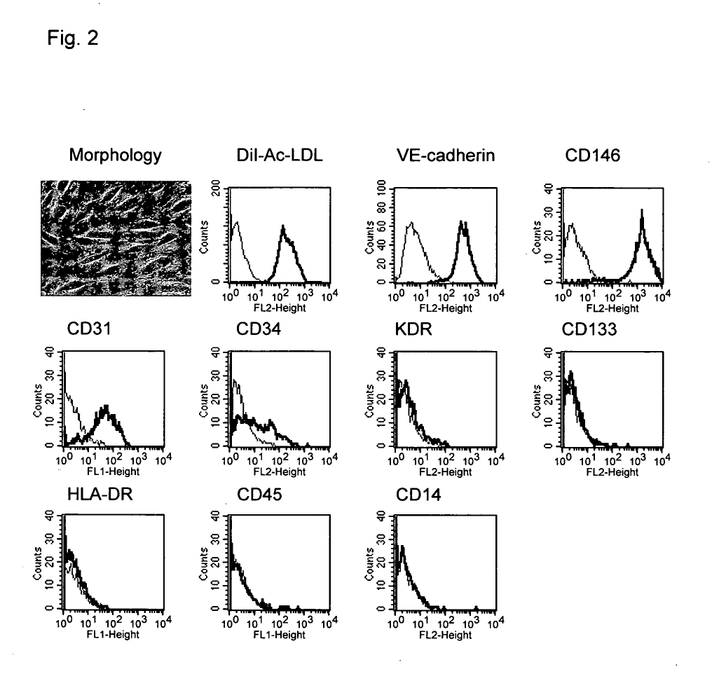

The adherent cells grew as monolayers of spindle-shaped flat cells (FIG. 2). The cells were able to incorporate DiI-Ac-LDL which was detected by fluorescence microscopy and flow cytometry (FIG. 2) and showed formation of tubuli in the Matrigel assay (see below). The cells were further characterized by flow cytometry with respect to the expression of various endothelial and hematopoietic surface markers and found to be uniformly positive for VE-cadherin (CD144), CD146 and CD31, which are typical of endothelial cells. The cells were uniformly negative for the hematopoietic markers CD45, CD14, CD133 and HLA-DR (FIG. 2). FACS analysis of five batches of EC showed that the cells obtained with this protocol were heterogenous concerning the expression of CD34 and KDR/VEGF-R2 (FIG. 2). CD34 expression was detected on a subset of 5-45% of cells, whereas KDR was weakly expressed by less than 5% of CBECs. The expression of the aforementioned cell surface markers appeared to be unchanged throughout cell expansion.

The data concerning expression of VE-cadherin, CD146, CD31, CD45, CD34 and KDR were confirmed by immunohistochemical analysis (IHC). Additionally, the expression of the endothelial marker von Willebrand factor (vWF) and and binding of Ulex europaeus agglutinin could be demonstrated by IHC.

Transduction Efficiency

In order to see whether CBECs can be transduced efficiently with lentiviral vectors, CBECs from four different pools of donors were transduced in a first series of experiments with the constructs pHR'SIN.cPPT-SEW encoding EGFP and cPPT-C(FVIIIΔB)IGWS encoding huBDD FVIII and EGFP at passage numbers three to eight and at multiplicities of infection (MOIs) of 10 and 100. The transduction efficiency was determined by detection of EGFP-positive cells at least at two different time points and not earlier than eight days after transduction.

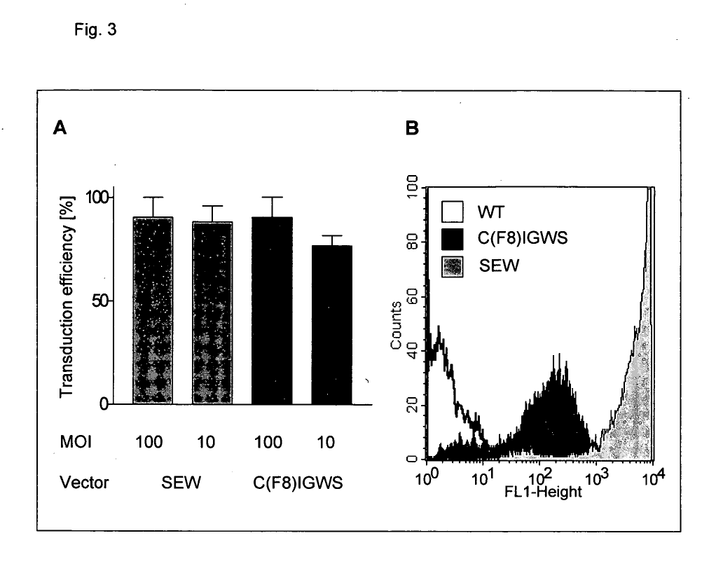

For the transduction of EC, MOIs of 10 yielded transduction efficiencies of 88.2%±7.6% (range 80.3%-95.6%) and 76.9%±5.0% (range 73.1%-84.2%) with pHR'SIN.cPPT-SEW and cPPT-C(FVIIIΔB)IGWS, respectively (FIG. 3A). When the MOIs were raised to 100, there was no statistically significant increase in transduction efficiencies (90.4%±9.5% and 90.6%±9.7%, p>0.05 for both vectors; FIG. 3A).

The percentage of EGFP-expressing cells as well as the EGFP expression level remained relatively unchanged during the culture period. FIG. 3B shows the EGFP expression of transduced vs. non-transduced cells 30 days after transduction with MOIs of 10 in a representative experiment.

Influence of Lentiviral Transduction on Cell Phenotype and Cell Proliferation

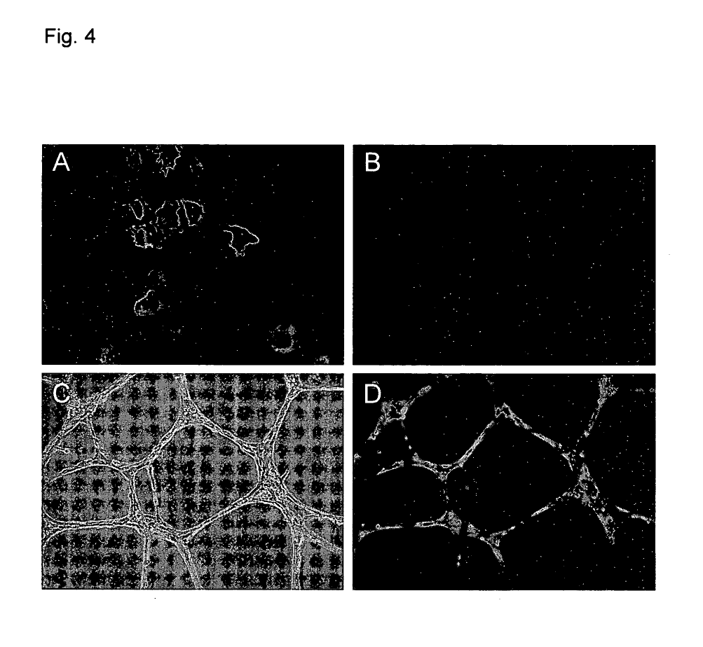

We observed some vector-mediated toxicity on the transduced EC which was apparent due to cell death during/after transduction (in particular with the higher MOI) and a lag phase before the cells returned to the proliferation rate of untransduced control cells as illustrated in FIG. 1. Hence, we wanted to investigate whether this was accompanied by alterations of cell phenotype between the transduced cells and the control cells. Cells were analyzed several weeks after transduction for the expression of CD146, CD34, KDR, and CD133 by flow cytometry and for the capability to incorporate DiI-Ac-LDL. The expression of the above mentioned cell surface markers was unchanged in comparison to untransduced cells, as was the uptake of DiI-Ac-LDL (FIGS. 4A&B). In addition, the CBECs retained their ability to form tubes in the Matrigel assay (FIGS. 4C&D) which is characteristic of mature and functional EC.

Quantification of FVIII Secretion by Chromogenic Assay and ELISA

The capacity of CBECs to express huBDD FVIII was investigated in seven independent experiments. Cells were transduced with the lentiviral construct cPPT-C(FVIIIΔB)IGWS encoding huBDD FVIII and EGFP and cultured as described above until senescence. FVIII secretion was quantified at various time points by seeding 5×10⁴ cells in 1 ml EGM-2 in 12 well plates and collecting the cell culture supernatants after 48 hours. Aliquots were stored at −80° C. until analysis using a chromogenic assay and an ELISA with a monoclonal antibody against the light chain as detection antibody. Cells were maintained from the transduction until they ceased to proliferate.

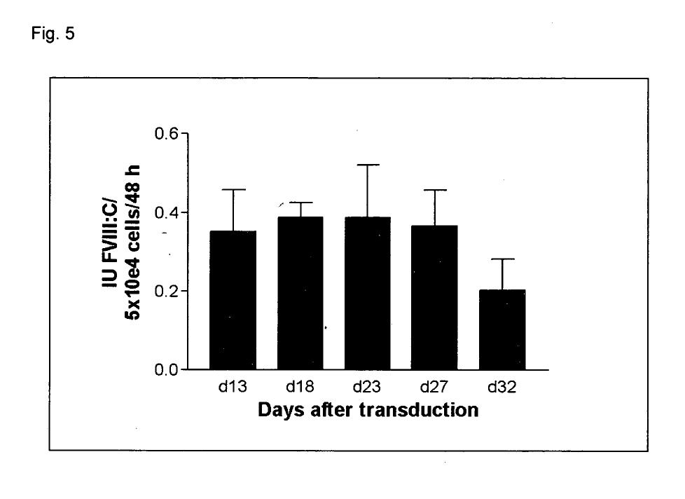

FVIII:C levels as measured by chromogenic assay were relatively constant until senescence. During the first four weeks after transduction of cells from three pools of donors, mean FVIII:C levels were 0.35-0.39 IU/5×10⁴ cells/48 h at an MOI of 10 corresponding to 7.0-7.8 IU/10⁶ cells/48 h (FIG. 5). FVIII:C secretion then decreased in all cultures which was accompanied by reduced proliferation and finally senescence. In a second set of experiments, the influence of the MOI on FVIII secretion was investigated. Raising the MOI from 10 to 100 lead only to a slight increase in FVIII:C secretion which was not statistically significant (p>0.05, n=4). Cells that were transduced with the control construct pHR'SIN.cPPT-SEW and untransduced cells did not secrete detectable amounts of FVIII:C (both <0.01 IU/5×10⁴ cells/48 h; n=7).

Determination of FVIII:Ag levels in the same EC supernatants yielded slightly higher FVIII:Ag concentrations with 0.45-0.66 IU/5×10⁴ cells/48 h at an MOI of 10 compared to FVIII:C (n=3). This resulted in mean ratios of FVIII:C/FVIII:Ag of 0.54-0.83 (n=3; Table 1).

In a control experiment, human umbilical vein endothelial cells (HUVECs) were transduced with the same huBDD FVIII-encoding vector. In six supernatants, the ratio of FVIII:C/FVIII:Ag was found to be 0.91±0.23. The latter ratio does not differ from the ratios that are characteristic of several hematopoietic cell lines transduced with a FVIII-encoding vector containing the same transgene cassette (Tonn et al., 2002).

Table 1. Specific activity of recombinant BDD FVIII in different human cell types. Hematopoietic cell lines, HUVECs and CBECs (cord-blood derived endothelial cells) were transduced with lentiviral vectors containing the same FVIII expression cassette. The specific activity was calculated as ratio between FVIII:Ag and FVIII:C in the supernatant of these cells and is given as mean values of at least three experiments.

| Transduced cell type | Mean ratio FVIII:C/FVIII:Ag |
|---|---|
| CBECs (Cord blood CD34⁺-derived | 0.54-0.83 |
| endothelial cells) | |
| HUVECs | 0.91 |
| Hematopoetic cell lines | 0.85-1.02 |
| (Tonn et al., 2002) | |

Characterisation of Secreted FVIII By Western Blot Analysis

In order to confirm the assumption that EC secrete correctly processed and hence active procoagulant FVIII, FVIII was enriched from cell culture supernatants by ultrafiltration using Vivaspin concentrators with a molecular weight cut-off of 30 kDa. Since the serum content of the EGM-2 did not allow effective concentration, EC were cultured over night without serum. Samples were concentrated up to 400fold and analyzed by ELISA, FVIII Western blotting and chromogenic assay.

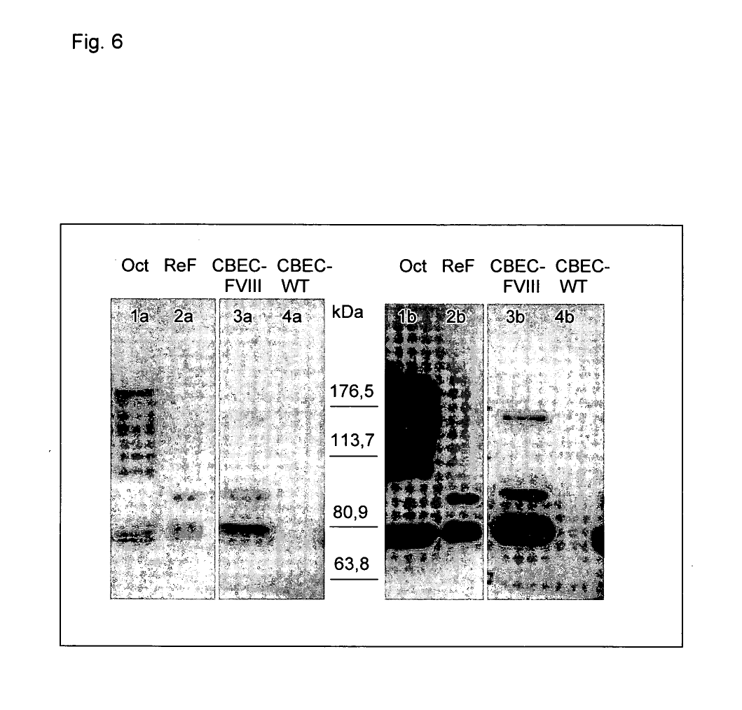

Concentrated supernatants of EC transduced with cPPT-C(FVIIIΔB)IGWS and of untransduced cells were subjected to SDS-PAGE and immunoblotting. As reference, plasma-derived FVIII (Octanate®) and recombinant B-domain deleted FVIII (ReFacto®) were used. Octanate® consists of a doublet of light chains of about 80 kDa and heavy chains of various sizes due to the different proteolytic steps involving the B domain in vivo. ReFacto® is composed of an 80 kDa doublet of light chains and a 90 kDa heavy chain. The deletion of the B domain used to express ReFacto® is almost identical to the deletion in the gene transfer vector that we used. Using a polyclonal anti-FVIII antibody, we therefore expected bands for the light chain at the same height in the EC-derived FVIII and the reference preparations and also comparable sizes of the heavy chain in EC-derived FVIII and ReFacto®. FIG. 6 demonstrates that this was indeed the case. After longer exposure, a weak band at 170 kDa appeared which indicates that the intracellular cleavage of FVIII into heavy chain and light chain might not have been complete. No FVIII protein could be detected in concentrated supernatants of untransduced cells.

Before immunoblotting, the FVIII:Ag concentrations of the EC-derived samples were determined by ELISA. The signal intensity found for EC-derived FVIII is in good agreement with the reference samples. With a chromogenic assay, we also confirmed that the concentrated supernatants exhibited procoagulant activity.

The invention provides a novel combination of cytokines for the differentiation of endothelial cells from CD34-positive cells, and it has been shown that umbilical cord blood was a feasible source of cells. After enrichment of CD34-positive cells from cord blood, the cultures containing VEGF, FGF-2, SCF and SCGF-β yielded adherent cells that had an endothelial phenotype and a very high, but limited proliferative potential. Previous studies have shown that endothelial cells can be derived and expanded considerably from progenitor cells in peripheral blood (Gehling et al., 2000; Lin et al., 2000) and bone marrow (Quirici et al., 2001), but umbilical cord blood has not been investigated in this respect. In the present study, the uniform expression of VE-cadherin (CD144), CD31, CD146 and LDL receptor, the absence of expression of CD45 and CD14, and the tube formation in the Matrigel assay demonstrated a clearly endothelial phenotype. A substantial subset of the cells retained the expression of CD34, whereas a surprisingly small percentage was only weakly KDR-positive. This pattern of cell surface markers has not been reported yet and differs from the studies of Gehling et al. (2000) and Lin et al. (2000) with respect to the expression of CD34, KDR, CD144 and CD31. The cells obtained with Gehling's protocol from CD133⁺ cells from G-CSF-mobilized peripheral blood were almost uniformly KDR-positive with only subsets expressing CD31, CD34 and CD144. Blood outgrowth endothelial cells (BOECs) from peripheral blood mononuclear cells that were differentiated according to Lin's protocol were uniformly positive for CD34, KDR, CD144 and CD31. The Matrigel assay has not been included in the other studies so that the cells cannot be compared with respect to the functional feature of tube formation. The cells also differed in their proliferative potential, which appeared to be huge in Lin's protocol (18 logs within 60 days) and relatively low in Gehling's study, whereas the protocol described supra allows for an intermediate expansion of cultures by 5 to 9 logs. After a total ex vivo culture period of several months with a constant cell phenotype, eventually senescence of cultures could be observed. Since excessive and uncontrollable proliferation in vivo as reported by Lin and coworkers on BOECs in immunodeficient mice might entail serious adverse effects, the limited expansion potential of the endothelial cells presented herein might actually be advantageous.

CBECs could also be useful target cells for hemophilia A gene therapy. A stably integrating, HIV-1-derived self-inactivating lentiviral vector with a B-domain deleted factor VIII cDNA and EGFP as marker gene was used for the transduction experiments. A moderate MOI of 10 proved to be sufficient to achieve high rates of transduction that were typically in the range of 75% to 95% for the FVIII/EGFP- or the EGFP-control construct. The transductions resulted in very high levels of FVIII secretion that were determined by a chromogenic assay and by ELISA. FVIII:C levels corresponded to 7.0-7.8 IU/10⁶ cells/48 h and were among the highest levels reported so far in other recombinant systems. Comparable levels of FVIII secretion were achieved in transduced HUVEC with the same vector, but 10-20fold lower levels in hematopoietic cell lines that were transduced with the same transgene cassette (Tonn et al., 2002). In addition, the level of FVIII secretion in HUVEC was found to be more than 10fold higher than reported by Chuah and colleagues who used a retroviral vector (Chuah et al., 1995).

The data on FVIII secretion were confirmed by a FVIII-specific ELISA, which yielded even slightly higher FVIII:Ag values so that the mean ratio of FVIII:C/FVIII:Ag was in the range of 0.54-0.83 (instead of about 1 as for hematopoietic cell lines and HUVEC). This reduction in specific activity could be caused by incomplete intracellular proteolysis because Western blotting of concentrated cell supernatant demonstrated that a part of the FVIII protein was secreted as a 170 kDa precursor with most of the FVIII being processed into heavy and light chains of 90 and 80 kDa as expected. But since FVIII activation in the plasma involves further proteolytic steps, the procoagulant activity in vivo of the CBEC-derived FVIII would probably be in the normal range.

The examples indicate that endothelial cells are particularly suited for the recombinant expression of FVIII and that the lentiviral vector allows for considerably more efficient recombinant FVIII expression than previously used viral or nonviral expression vectors. Although transduction experiments at an MOI of 10 resulted in some cell death during/after incubation with vector and a short lag phase in proliferation compared to untransduced cells, phenotypical alterations or a net reduction or increase of expansion of the transduced CBECs could not be detected. The levels of transgene expression as determined by a FVIII:C-specific chromogenic assay or by flow cytometry for EGFP remained relatively stable until the cells ceased to proliferate. Hence, the vector system chosen in the examples was not only highly efficient, but also safe in this preliminary in vitro analysis.

Endothelial cells derived from cord blood are an attractive autologous source of cells for hemophilia A gene therapy. It is known that for about two thirds of all hemophilia A patients, the underlying mutation is inherited and therefore their FVIII deficiency is predictable based on the family history. In these cases, the CD34-positive cells from umbilical cord blood that would usually be discarded could be used for endothelial differentiation cultures. For adolescent or adult patients, the protocol presented in the examples for CBECs can be adapted to CD34-positive cells from peripheral blood or bone marrow.

The invention therefore also relates to a method for the preparation of endothelial cells expressing a protein, e.g. a blood coagulation factor, comprising a) contacting in vitro human endothelial precursor cells with the growth factors VEGF, bFGF, SCF and SCGF-β; and b) transducing the cells with DNA encoding the protein. The endothelial precursor cells may be derived from bone marrow, peripheral blood or cord blood. The preferred embodiments of this method correspond to the preferred embodiments of the methods of the invention described supra.

After lentiviral transduction at an early time-point, the culture could be expanded and analyzed for efficient FVIII secretion and safety features such as the absence of replication-competent lentivirus. Frozen batches of cells could be stored and used for multiple injections during life.

The senescence observed in all in vitro cultures so far suggest a moderate number of population doublings in vivo and therefore the requirement for repeated cell infusions. This scenario might be preferable to the in vivo proliferation of FVIII-transfected BOECs that were shown to engraft in the spleen and in bone marrow of immunodeficient mice after injections into the tail vein without accompanying conditioning regimen (Lin et al., 2002). On the basis of the data published so far, long-term complications such as replacement of bone marrow by BOECs leading to hematological abnormalities or enhanced risk of thrombosis will have to be considered and must be ruled out before any therapeutic application of these cells unless their in vivo proliferation can be controlled.

The present invention provides the use of endothelial cells, in particular CBECs for the gene therapy of hemophilia A and other congenital disorders that are characterized by the absence of plasma proteins such as factor IX, von Willebrand factor or α₁-antitrypsin.

### REFERENCES

ANDREWS, J. L., WEAVER, L., KALEKO, M., CONNELLY, S. (1999). Efficient adenoviral vector transduction and expression of functional human factor VIII in cultured primary human hepatocytes. Haemophilia 5, 160-168.

ASAHARA, T., MUROHARA, T., SULLIVAN, A., SILVER, M., VAN DER ZEE, R., LI, T., WITZENBICHLER, B., SCHATTEMAN, G., ISNER, J. M. (1997). Isolation of putative progenitor endothelial cells for angiogenesis. Science 275, 964-967.

ASAHARA, T., ISNER, J. M. (2002). Endothelial progenitor cells for vascular regeneration. J. Hematoth. Stem Cell Res. 11, 171-178.

CHARNEAU, P., ALIZON, M., CLAVEL, F. (1992). A second origin of DNA plus-strand synthesis is required for optimal human immunodeficiency virus replication. J. Virol. 66, 2814-2820.

CHUAH, M. K. L., VANDENDRIESSCHE, T., MORGAN, R. A. (1995). Development and analysis of retroviral vectors expressing human factor VIII as a potential gene therapy for hemophilia A. Hum. Gene Ther. 6, 1363-1377.

CHUAH, M. K. L., BREMS, H., VANSLEMBROUCK, V., COLLEN, D., VANDENDRIESSCHE, T. (1998). Bone marrow stromal cells as targets for gene therapy of hemophilia A. Hum. Gene Ther. 9, 353-365.

CHUAH, M. K. L., COLLEN, D., VANDENDRIESSCHE, T. (2001). Gene therapy for hemophilia. J. Gene Med. 3, 3-20.

DEMAISON, C., PARSLEY, K., BROUNS, G., SCHERR, M., BATTMER, K., KINNON, C., GREZ, M., THRASHER, A. J. (2002). High-level transduction and gene expression in hematopoietic repopulating cells using a human immunodeficiency virus type 1-based lentiviral vector containing an internal spleen focus forming virus promoter. Hum. Gene Ther. 13, 803-813.

DO, H., HEALEY, J. F., WALLER, E. K., LOLLAR, P. (1999). Expression of factor VIII by murine liver sinusoidal endothelial cells. J. Biol. Chem. 274, 19587-19592.

DWARKI, V. J., BELLONI, P., NIJJAR, T., SMITH, J., COUTO, L., RABIER, M., CLIFT, S., BERNS, A., COHEN, L. K. (1995). Gene therapy for hemophilia A: production of therapeutic levels of human factor VIII in vivo in mice. Proc. Natl. Acad. Sci. U.S.A. 92, 1023-1027.

EVANS, G. L., MORGAN, R. A. (1998). Genetic induction of immune tolerance to human clotting factor VIII in a mouse model for hemophilia A. Proc. Natl. Acad. Sci. U.S.A. 95, 5734-5739.

FAKHARZADEH, S. S., ZHANG, Y., SARKAR, R., KAZAZIAN, H. H. JR. (2000). Correction of the coagulation defect in hemophilia A mice through factor VIII expression in skin. Blood 95, 2799-2805.

FOLLENZI, A., AILLES, L. E., BAKOVIC, S., GEUNA, M., NALDINI, L. (2000). Gene transfer by lentiviral vectors is limited by nuclear translocation and rescued by HIV-1 pol sequences. Nat. Genet. 25, 217-222.

FOLLENZI, A., NALDINI, L. (2002). Generation of HIV-1 derived lentiviral vectors. Methods Enzymol. 346, 454-465.

GARCIA-MARTIN, C., CHUAH, M. K. L., VAN DAMME, A., ROBINSON, K. E., VANZIELEGHEM, B., SAINT-REMY, J. M., GALLARDO, D., OFOSU, F. A., VANDENDRIESSCHE, T., HORTELANO, G. (2002). Therapeutic levels of human factor VIII in mice implanted with encapsulated cells: potential for gene therapy of hemophilia A. J. Gene Med. 4, 215-223.

GEHLING, U. M., ERGÜN, S., SCHUMACHER, U., WAGENER, C., PANTEL, K., OTTE, M., SCHUCH, G., SCHAFHAUSEN, P., MENDE, T., KILIC, N., KLUGE, K., SCHÄFER, B., HOSSFELD, D. K., FIEDLER, W. (2000). In vitro differentiation of endothelial cells from AC133-positive progenitor cells. Blood 95, 3106-3112.

GRANT, M. B., MAY, W. S., CABALLERO, S., BROWN, G. A. J., GUTHRIE, S. M., MAMES, R. N., BYRNE, B. J., VAUGHT, T., SPOERRI, P. E., PECK, A. B., SCOTT, E. W. (2002). Adult hematopoietic stem cells provide functional hemangioblast activity during retinal neovascularization. Nat. Med. 6, 607-612.

GREENGARD, J. S., JOLLY, D. J. (1999). Animal testing of retroviral-mediated gene therapy for factor FVIII deficiency. Thromb. Haemost. 82, 555-561.

HOEBEN, R. C., VAN DER JAGT, R. C., SCHOUTE, F., VAN TILBURG, N. H., VERBEET, M. P., BRIET, E., VAN ORMONDT, H., VAN DER EB, A. J. (1990). Expression of functional factor VIII in primary human skin fibroblasts after retrovirus-mediated gene transfer. J. Biol. Chem. 265, 7318-7323.

HOEBEN, R. C., EINERHAND, M. P., BRIET, E., VAN ORMONDT, H., VALERIO, D., VAN DER EB, A. J. (1992). Toward gene therapy in haemophilia A: retrovirus-mediated transfer of a factor VIII gene into murine hematopoietic progenitor cells. Thromb. Haemost. 67, 341-345.

HOLLESTELLE, M. J., THINNES, T., CRAIN, K., STIKO, A., KRUIJT, J. K., VAN BERKEL, T. J. C., LOSKUTOFF, D. J., VAN MOURIK, J. A. (2001). Tissue distribution of FVIII gene expression in vivo—a closer look. Thromb. Haemost. 86, 855-861.

HOOTS, W. K. (2001). History of plasma-product safety. Transfus. Med. Rev. 15 (2 suppl. 1), 3-10.

IWAGURO, H., YAMAGUCHI, J., KALKA, C., MURASAWA, S., MASUDA, H., HAYASHI, S., SILVER, M., LI, T., ISNER, J. M., ASAHARA, T. (2002). Endothelial progenitor cell VEGF gene transfer for vascular regeneration. Circulation 105, 732-738.

JACKSON, K. A., MAJKA, S. M., WANG, H., POCIUS, J., HARTLEY, C. J., MAJEWSKY, M. W., ENTMAN, M. L., MICHAEL, L. H., HIRSCHI, K. K., GOODELL, M. A. (2001). Regeneration of ischemic cardiac muscle and vascular endothelium by adult stem cells. J. Clin. Invest. 107, 1395-1402.

KAUFMAN, R. J. (1999). Advances toward gene therapy for hemophilia at the millennium. Hum. Gene. Ther. 10, 2091-2107.

LIN, Y., WEISDORF, D. J., SOLOVEY, A., HEBBEL, R. P. (2000). Origins of circulating endothelial cells and endothelial outgrowth from blood. J. Clin. Invest. 105, 71-77.

LIN, Y., CHANG, L., SOLOVEY, A., HEALEY, J. F., LOLLAR, P., HEBBEL, R. P. (2002). Use of blood outgrowth endothelial cells for gene therapy for hemophilia A. Blood 99, 457-462.

LOZIER, J. N., METZGER, M. E., DONAHUE, R. E., MORGAN, R. A. (1999). Adenovirus-mediated expression of human coagulation factor IX in the rhesus macaque is associated with dose-limiting toxicity. Blood 94, 3968-3975.

MANNUCCI, P. M., GIANGRANDE, P. L. F. (2000). Choice of replacement therapy for hemophilia: recombinant products only? Hematol. J. 1, 72-76.

MANNUCCI, P. M., TUDDENHAM, E. G. D. (2001). The hemophilias—from royal genes to gene therapy. N. Engl. J. Med. 344, 1773-1779.

MUROHARA, T. (2001). Therapeutic vasculogenesis using human cord blood-derived endothelial progenitors. Trends Cardiovasc. Med. 11, 303-307.

MURRAY, P. D. F. (1932). The development in vitro of blood of the early chick embryo. Proc. Roy. Soc. 111, 497-521.

MYERS, G., JOSEPHS, S. F., BERZOFSKY, J. A., RABSON, A. B., SMITH, T. F., WONG-STAAL, F. (eds.) (1989). Human retroviruses and AIDS 1989—a compilation and analysis of nucleic acid and amino acid sequences. Los Alamos National Laboratory, Los Alamos, N. Mex.

QUIRICI, N., SOLIGO, D., CANEVA, L., SERVIDA, F., BOSSOLASCO, P., LAMBERTENGHI DELILIERS, G. (2001). Differentiation and expansion of endothelial cells from human bone marrow CD133⁺ cells. Br. J. Haematol. 115, 186-194.

REDING, M. T., Wu, H., KRAMPF, M., OKITA, D. K., DIETHELM-OKITA, B. M., KEY, N. S., CONTI-FINE, B. M. (1999). CD4⁺ T cell response to FVIII in hemophilia A, acquired hemophilia, and healthy subjects. Thromb. Haemost. 82, 509-515.

ROSENBERG, J. B., GREENGARD, J. S., MONTGOMERY, R. R. (2000). Genetic induction of a releasable pool of factor VIII in human endothelial cells. Arterioscler. Thromb. Vasc. Biol. 20, 2689-2695.

ROTH, D. A., TAWA, N. E. JR., O'BRIEN, J. M., TRECO, D. A., SELDEN, R. F. (2001). Nonviral transfer of the gene encoding coagulation factor VIII in patients with severe hemophilia. N. Engl. J. Med. 344, 1735-1742.

SABIN, F. (1920). Studies on the origin of blood vessels and of red blood corpuscules as seen in the living blastoderm of chicks during the second day of incubation. Carnegie Inst. Pub. 9-36, 214-262.

SIRVEN, A., PFLUMIO, F., ZENNOU, V., TITEUX, M., VAINCHENKER, W., COULOMBEL, L., DUBART-KUPPERSCHMITT, A., CHARNEAU, P. (2000). The human immunodeficiency virus type-i central DNA flap is a crucial determinant for lentiviral vector nuclear import and gene transduction of hematopoietic stem cells. Blood 96, 4103-4110.

TAGLIAVACCA, L., WANG, Q., KAUFMAN, R. J. (2000). ATP-dependent dissociation of non-disulfide-linked aggregates of coagulation factor VIII is a rate-limiting step for secretion. Biochemistry 39, 1973-1981.

TEITEL, J. M. (2000). Viral safety of haemophila treatment products. Ann. Med. 32, 485-492.

TONN, T., HERDER, C., BECKER, S., SEIFRIED, E., GREZ, M. (2002). Generation and characterization of human hematopoietic cell lines expressing factor VIII. J. Hematoth. Stem Cell Res. 11, 695-704.

VANDENDRIESSCHE, T., COLLEN, D., CHUAH, M. K. L. (2001). Viral vector-mediated gene therapy for hemophilia. Curr. Gene Ther. 1, 301-315.

WHITE II, G. C., GREENWOOD, R., ESCOBAR, M., FRELINGER, J. A. (2000). Hemophilia factor VIII therapy. Immunological tolerance. A clinical perspective. Haematologica 85 (10 suppl.), 113-116.

WHITE II, G. C. (2001). Gene therapy in hemophilia: clinical trials update. Thromb. Haemost. 86, 172-177.

WION, K. L., KELLY, D., SUMMERFIELD, J. A., TUDDENHAM, E. G., LAWN, R. M. (1985). Distribution of FVIII mRNA and antigen in human liver and other tissues . Nature 317, 726-729.

YANG, Y., JOOSS, K. U., SU, Q., ERTL, H. C., WILSON, J. M. (1996). Immune responses to viral antigens versus transgene product in the elimination of recombinant adenovirus-infected hepatocytes in vivo. Gene Ther. 3, 137-144.

ZELECHOWSKA, M. G., VAN MOURIK, J. A., BRODNIEWICZ-PROBA, T. (1985). Ultrastructural localization of factor VIII procoagulant antigen in human liver hepatocytes. Nature 317, 729-730.

ZENNOU, V., PETIT, C., GUETARD, D., NEHRBASS, U., MONTAGNIER, L., CHARNEAU, P. (2000). HIV-1 genome nuclear import is mediated by a central DNA flap. Cell 101, 173-185.

ZUFFEREY, R., NAGY, D., MANDEL, R. J., NALDINI, L., TRONO, D. (1997). Multiply attenuated lentiviral vector achieves efficient gene delivery in vivo. Nature Biotechnol. 15, 871-875.

While the invention has been described in detail with reference to exemplary embodiments thereof, it will be apparent to one skilled in the art that various changes can be made, and equivalents employed, without departing from the scope of the invention. Each of the aforementioned documents, including the foreign priority document, EP 03008076.6, is incorporated by reference herein in its entirety.

1. A method for the preparation of human endothelial cells expressing a protein, comprising:
a) contacting in vitro human cord blood-derived endothelial precursors cells and/or bone marrow-derived endothelial precursor cells with at least one growth factor, wherein the at least one growth factor promotes the differentiation of endothelial precursor cells into mature endothelial cells; and
b) transducing the mature endothelial cells with DNA encoding the protein, or transducing, prior to step a), the endothelial precursor cells with DNA encoding the protein.

2. A method according to claim 1, wherein the human cord blood-derived endothelial precursor cells express at least one cell surface marker selected from the group consisting of CD34, AC133, CD146, and FGF1-R.

3. A method according to claim 1, further comprising enriching cells selected from the group consisting of CD34-positive cells, AC133-positive cells, CD146-positive cells, FGF1-R-positive cells, and combinations thereof from cord blood prior to step a) and b).

4. A method according to claim 1 wherein a retroviral vector is used to transduce the cells.

5. A method according to claim 4, wherein said retroviral vector is a lentiviral vector.

6. A method according to claim 1 wherein the protein is a blood coagulation factor.

7. A method according to claim 6, wherein the blood coagulation factor is human factor VIII.

8. A population of human endothelial cells obtainable by a process according to claim 1.

9. A population of human endothelial cells derived from cord blood wherein at least 10% of the cells contain recombinant DNA encoding a protein.

10. A population of human endothelial cells according to claim 8 wherein the protein is human blood coagulation factor VIII or IX.

11. A population of human endothelial cells according to claim 8 wherein the recombinant DNA encoding a protein encodes a mutein of human blood coagulation factor VIII.

12. A population of human endothelial cells according to claim 11 wherein the mutein of human factor VIII at least partially lacks the B domain of wild type factor VIII.

13. A population of human endothelial cells according to claim 8, wherein the recombinant DNA encoding a protein is a modified factor VIII cDNA, wherein
(i) at least part of the B-domain of the wild-type factor VIII cDNA has been deleted,
(ii) at least one intron has been inserted into at least one location of the factor VIII cDNA, and/or
(iii) at least one nucleotide of the wild-type cDNA sequence of human factor VIII is substituted.

14. A population of human endothelial cells according to claim 8, wherein at least 75% of the cells express at least one marker selected from the group consisting of CD144, CD31, CD146, and LDL-receptor.

15. A population of human endothelial cells according to claim 8, wherein at least 75% of the cells endogenously express VWF.

16. An immortalized cell line generated from a population of human endothelial cells according to claim 8.

17. A method for the production of a protein, comprising
a) culturing a population of human endothelial cells according to claim 8 under suitable conditions; and
b) isolating the protein from the cell culture medium.

18. A method for the production of a protein of claim 17, wherein the protein is a blood coagulation factor.

19. A method according to claim 17, wherein the protein is blood coagulation factor VIII and step b) comprises purifying blood coagulation factor VIII in the presence of von Willebrand factor.

20. A method according to claim 17, wherein the protein is subjected to virus inactivation treatment.

21. The method according to claim 20, wherein the protein is a blood coagulation factor.

22. A method for the production of a protein, comprising
a) culturing a cell line according to claim 16 under suitable conditions; and
b) isolating the protein from the cell culture medium.

23. A method for the production of a protein of claim 22, wherein the protein is a blood coagulation factor.

24. A method according to claim 22, wherein the protein is blood coagulation factor VIII and step b) comprises purifying blood coagulation factor VIII in the presence of von Willebrand factor.

25. A method according to claim 22, wherein the protein is subjected to virus inactivation treatment.

26. The method according to claim 25, wherein the protein is a blood coagulation factor.

27. A method of treating hemophilia A or hemophilia B in an individual comprising administering to said individual a medicament comprising a population of human endothelial cells according to claim 8.

28. The method according to claim 27, wherein said human endothelial cells secrete human blood coagulation factor VIII and said individual suffers from hemophilia A.

29. A method of treating a disorder affecting the cardiovascular system in an individual comprising administering to said individual a population of human endothelial cells according to claim 8.

30. The method according to claim 29, wherein the disorder is selected from the group consisting of cardiac failure, acute heart disease, and chronic heart disease.

31. A pharmaceutical composition comprising a population of human endothelial cells according to claim 8.


SEQUENCE LISTING (3 sequences)

SEQ ID NO: 1 - DNA, 25 bp, Artificial Sequence
  Primer 1
ccatcgatacaaatggcattcatcc

SEQ ID NO: 2 - DNA, 36 bp, Artificial Sequence
  Primer 2
ccatcgatctcgagccaaagtggatctctgctgtcc

SEQ ID NO: 3 - protein, 2351 aa, homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY